(19) United States
(12) Patent Application Publication
(10) Pub. No.: US 2009/0275810 A1
(43) Pub. Date: Nov. 5, 2009

(54) PORTABLE MODULAR PC BASED SYSTEM FOR CONTINUOUS MONITORING OF BLOOD OXYGENATION AND RESPIRATORY PARAMETERS

(75) Inventors: Eric J. Ayers, Alliquippa, PA (US); Eric W. Starr, Allison Park, PA (US)

Correspondence Address: BLYNN L. SHIDELER;THE BLK LAW GROUP, 3500 BROKKTREE ROAD, SUITE 200, WEXFORD, PA, 15090, US

(73) Assignee: STARR Life Sciences Corp., Oakmont, PA (US)

(21) Appl. No.: 12/485,902

(22) Filed: Jun. 16, 2009

Related U.S. Application Data
(63) Continuation-in-part of application No. 12/434,599, filed on May 1, 2009.
(60) Provisional application No. 61/073,003, filed on Jun. 16, 2008.
(60) Provisional application No. 61/049,451, filed on May 1, 2008.

(51) Int. Cl. A61B 5/00; G08B 23/00

(52) U.S. Cl. 600/301; 340/573.1

(57) ABSTRACT

A portable modular kiosk based physiologic sensor system for clinical and research applications configured to simultaneously utilize multiple sensors with cross checking and cross calculation of physiologic parameters and configured for continuous monitoring of blood oxygenation and respiratory parameters.

### RELATED APPLICATIONS

The present application claims the benefit of U.S. Provisional Patent Application Ser. No. 61/073,003 entitled “Apparatus for Continuous Monitoring of Blood Oxygenation and Respiratory Parameters” filed Jun. 16, 2008.

The present application is a continuation in part of U.S. patent application Ser. No. 12/434,599 entitled “Portable Modular Kiosk Based Physiologic Sensor System with Display and Data Storage for Clinical and Research Applications including Cross Calculating and Cross Checked Physiologic Parameters Based Upon Combined Sensor Input” filed May 1, 2009.

U.S. patent application Ser. No. 12/434,599 claims the benefit of U.S. Provisional Patent Application Ser. No. 61/049,451 entitled “Portable Modular Kiosk Based Physiologic Sensor System for Clinical and Research Applications Configured to Simultaneously Utilize Multiple Sensors with Cross Checking and Cross Calculation of Physiologic Parameters” filed May 1, 2008.

### BACKGROUND OF THE INVENTION

1. Field of the Invention

The present invention relates to a portable, modular physiologic sensor system with display and data storage for clinical and research applications, in particular to small mammal applications, such as neonates in humans and rats and mice in research applications, for continuous monitoring of blood oxygenation and respiratory parameters.

2. Background Information

The present invention relates to monitoring of physiologic parameters of a patient or subject, in particular an animal patient or subject, such as a small mammal, such as neonates in humans and rats and mice in research applications. The following definitions will be helpful in explaining the known background elements that are helpful for understanding the present invention. Physiologic parameters are measured with physiologic sensors that typically, but not always, contact the patient or subject. The term patient is appropriate in the medical fields for both the human medical field and animal veterinarian fields. The term subject is appropriate in the research field, and the term subject will apply to human and non-human applications.

The list of physiologic sensors is large and constantly growing. A representative list of physiologic sensors known in the art include blood pressure sensors, blood flow sensors, blood glucose sensors, blood cholesterol sensors, heart sound sensors, EMG sensors, EEG sensors, EKG sensors, EOG sensors, pulse sensors, oxygenation sensors, blood perfusion sensors, respiration monitors, temperature sensors, blood gas sensors, motion sensors, strain gauges, body position sensors, and limb motion sensors.

A “kiosk’ within this application, sometimes called an electronic kiosk, computer kiosk or interactive kiosk, houses a computer terminal that often employs custom kiosk software designed to function, hopefully flawlessly, while preventing users from accessing system functions. “Kiosk mode” is a euphemism for such a mode of software operation. Computerized kiosks may store data locally, or retrieve it from a computer network. Some common computer kiosks provide a free, informational public service while others common computer kiosks serve a commercial purpose. Touch-screens, trackballs, computer keyboards, and pushbuttons are all typical input devices for interactive computer kiosk.

The “personal computer” or simply “PC” is a term that is so often used it seems unlikely, at first, to require formal definition. However the precise scope of the term is sometimes vague. A PC is a computer whose size, and capabilities (and some have said price) make it useful for individuals, intended to be operated directly by an end user and capable of performing a variety of general purpose tasks, with no intervening computer operator. The PC may be a home computer, or may be found in an office, a medical facility or a research lab. The PC may often be connected to a local area network. The distinguishing characteristics of a PC are that the computer is primarily used, interactively, by one person at a time. This is opposite to the batch processing or time-sharing models which allowed large expensive systems to be used by many people, usually at the same time, or large data processing systems which required a full-time staff to operate. The PC can come in desktop models, notebook models, handheld models, and hybrids of these.

A “notebook computer”, or simply “notebook” within this application, is an extremely lightweight PC. Notebook computers typically weigh less than 6 pounds and are small enough to fit easily in a briefcase. Aside from size and portability, the principal difference between a notebook computer and a non-notebook personal computer (e.g. a desktop computer) is the display screen. Notebook computers use a variety of techniques, known as flat-pane technologies, to produce a lightweight and non-bulky display screen. Laptop computers and tablet PCs are two types of notebook computers. Usually all of the interface hardware needed to operate the notebook computer, such as parallel and serial ports, graphics card, sound channel, etc., are built in to a single unit. Most notebook computers contain batteries to facilitate operation without a readily available electrical outlet.

A “laptop computer”, or simply laptop, is, within this application, a subset of notebooks. A laptop will have a display and separate keyboard interface (e.g. “qwerty” keyboard), with the keyboard and the display typically hinged together. The term Laptop is sometimes used more broadly and equated with notebooks, but the term will have a narrower definition within this application.

A “Tablet PC” is a notebook, also called a panel computer, and was first introduced by Pen Computing in the early 90s with their PenGo Tablet Computer and popularized by Microsoft. The touch-screen or “graphics tablet/screen hybrid technology” technology of the tablet PHOTOCHROMIC allows the user to operate the computer with a stylus or digital pen, or a fingertip, instead of a keyboard or mouse. The tablet PC is particularly well suited to operate in Kiosk mode in light of the built in user interface provided with the tablet PC.

The input/output ports of a personal computer refer to the communications links through which the personal computers send and receive information, which generally include serial ports, parallel ports, wireless links or connectors (such as WI-FL and Bluetooth), and universal serial bus (USB) ports. In addition, some laptops have expansion slots for PCMCIA standard adaptor cards (Type I and Type II) that also form input/output ports.

In the clinical fields, physiologic parameters of subjects are typically viewed through a “medical monitor” that is defined as an automated medical device that senses a patient's vital signs through an associated sensor and displays the results. Medical monitors are typically highly specialized and suited solely for the designated monitoring tasks. The modern trend is to have multi-parameter medical monitors that can track and display different vital signs on a common display. This specialization has limited the applicability of many of these devices in the research applications. Further, this specialization has, in some applications, limited the portability of the medical monitor, limiting the applications to the hospital or clinic environment and resulting in the device being impractical for certain portable applications such as needed in the veterinary fields.

In the research field, physiologic parameters of subjects can, of course, be viewed through a “medical monitor” in the same manner as in current clinical fields. The assignee of this application has developed laptop and desktop PC based physiologic sensors that have been embraced by researchers. These devices, such as the MOUSE OX™ brand pulse oximeters, allow researchers to collect data on the physiologic parameters of the subjects such as mice on the researcher's laptop or desktop PC and to work with the data on their laptop or desktop PC. These laptop and desktop PC based physiologic sensor systems could, in theory, be operated in the clinical environments. However, the current laptop and desktop PC based physiologic sensor systems have not been widely adopted in the clinical fields because (1) there is a more significant space restriction associated with many clinical applications (i.e. there is no available desktop space and thus the system would require its own cart or stand), and (2) there is a need in clinical environments to avoid the program start up and selection procedures associated with general operating PCs as clinical technician are less inclined to work through an operating system to view and/or record the physiologic parameters.

A physiologic sensor within the meaning of this specification is a sensor that measures a parameter related to a physical characteristic of a living subject, such as a human or animal. The types of physiologic sensors include, for example, blood pressure sensors, blood flow sensors, blood glucose sensors, blood cholesterol sensors, heart sound sensors, EMG sensors, EEG sensors, EKG sensors, EOG sensors, pulse sensors, oxygenation sensors, pulse-oximetry sensors, blood perfusion sensors, respiration sensors (both pressure, flow and rate), temperature sensors, additional blood gas sensors (such as nitrogen partial pressure, carbon dioxide partial pressure, carbon monoxide partial pressure, oxygen partial pressure, and pH level), motion sensors, strain gauges, body position sensors, limb motion sensors and the like.

Respiratory sensors are a subset of physiologic sensors and refer to those sensors primarily measuring physical parameters of a subject indicative of respiration of the subject. A ventilation based sensor is, for example, a respiratory sensor. Cardiac sensors are a subset of physiologic sensors and refer to those sensors primarily measuring physical parameters of a subject indicative of cardiac function of the subject. A pulse oximeter is, for example, a cardiac sensor. However, as noted in detail below, a respiratory sensor can provide signals indicative of other physiologic parameters outside of respiration and a cardiac sensor can provide signals indicative of non-cardiac functions. For example the pulse oximeter can be used to calculate breathing rates.

U.S. Published Patent Application 2006-0264762 discloses a personal computer (PC) based physiologic monitor system that includes a personal computer having a display and an input/output port for attachment to an external device. The PC based system also includes a physiologic sensor coupled to the personal computer through the input/output port so that a modified output of the physiologic sensor is graphically displayed on the display. A controller, a portion of which is disposed in the personal computer, modifies the output of the physiologic sensor and provides a feedback control signal for modifying the output of the physiologic sensor. This disclosure is incorporated herein by reference, portions of which are repeated below for convenience.

FIG. 1 is a schematic prior art respiratory system 10 for outpatient surgery of U.S. Published Patent Application 2006-0264762. Three common systems for supplying sedation anesthesia to the patient 12 include an intravenous supply system for anesthesia such as shown in FIG. 1, a respiratory system coupled to the patient, such as shown in FIG. 2, and needle and syringe injection (not shown). In the intravenous supply system of FIG. 1 the sedentary aesthesia is provided in an appropriate solution in an I.V. bag 14 on a conventional stand 16. As noted, a needle and syringe could also be used to supply intravenous sedentary anesthesia to the patient 12 through simple, periodic injections. The respiratory system 10 of U.S. Published Patent Application 2006-0264762 includes a ventilatory system coupled to a patient 12. Specifically the ventilatory system has a controlled blower motor 20, having a respiratory gas intake and power supply (not shown) and a respiratory gas output coupled to the patient 12 through tubing 22 and mask 24. The mask 24 may be replaced by nasal canella or other respiratory patient couplings as desired. The mask 24 or tubing 22 includes a vent 26 for a patients exhaling, as generally known in the art. The respiratory system 10 includes a respiratory sensor 28 coupled to the patient 12 and adapted to detect a respiration parameter of the patient 12. In FIG. 1 the sensor 28 is attached to the patient 12 through the tubing 22, and in this configuration it may be a pressure or flow sensor for detecting the respiration parameters of the patient 12. The sensor 28 could be placed on the mask 24, at the vent 26, or even on the blower motor 20, and obtain signals indicative of the patient's respiration parameters. The sensor 28 may be placed directly on the patient 12 as well. The specific type and the location of the sensor 28 can vary, provided that the sensor provides an output indicative of the patient's respiration parameters (i.e. at least the time of and preferably an indication of how much respiratory volume the patient is receiving with each breath). In the respiratory system 10 the blower motor 20 and the respiratory sensor 28 are coupled to a central controller that is in the form of a lap-top computer 30. The sensor 28 is coupled to the computer 30 through an amplifier 32 to prove a meaningful signal to the computer 30. The coupling between the amplifier 32 and the computer 30, shown as link 34, may be a hardwire connection or a wireless connection. In a similar fashion, the coupling between the blower motor 20 and the computer 30, shown as link 36, may be a hardwire connection or a wireless connection. Where the links 34 are hardwire connections, it is preferred that they couple to conventional existing ports of the laptop computer 30. The respiratory system 10 additionally includes further physiologic sensors coupled to the patient 12. Specifically a pulse oximeter sensor 40 is attached to the patient 12 and coupled to the computer 30 through an amplifier 42 and link 44. The link 44 between the amplifier 42 and the computer 30, shown discussed above, may also be a hardwire connection or a wireless connection. The addition of physiologic sensors, such as sensors 28 and 40, allows the computer to be a physiologic monitor graphically displaying the sensed parameters of the patient, as will be described in detail hereinafter. The sensors for this physiologic monitor are not limited to respiratory, pulse and blood oxygenation as shown in FIGS. 1 and 2, but may further include a blood pressure sensor, a blood flow sensor, a blood glucose sensor, a blood cholesterol sensor, a heart sound sensor, an EMG sensor, an EEG sensor, an EKG sensor, an EOG sensor, a blood perfusion sensor, a temperature sensor, a blood gas sensor, a motion sensor, a strain gauge, a body position sensor, a limb motion sensor, and any combinations thereof.

The respiratory system 10′ of FIG. 2 is similar to system 10 of FIG. 1 and is also described U.S. Published Patent Application 2006-0264762. Prior art FIG. 2 differs in the system for supplying sedation anesthesia to the patient 12. Inhaled anesthesia agents are used in the embodiment of FIG. 2, which are supplied to the blower motor 20 through anesthesia gas supply 50 and input 52. When using inhaled agents for anesthesia the ventilatory system cannot vent to the room, or it could adversely affect the caregivers. Therefore a closed system is created where vent 26 is replaced with a one way T coupling 26′ leading, through tubing 54, to a CO₂/anesthesia scrubber 56 that can vent harmless material or return the scrubbed respiratory gasses to the input 52 through tubing 58. A source of oxygen 60 is coupled to the input 52 through tubing 62 to supply oxygen to the closed system. An oxygen sensor 64 may be provided on input 52 (or else ware on the closed system) and coupled to the controller 30 through link 66. The link 66 between the sensor 64 (which may have an amplifier—not shown) and the computer 30, shown discussed above, may also be a hardwire connection or a wireless connection. As a closed respiratory system it is important to track the oxygen level received by the patient 12.

There remains a need in the art to for a simple to simple to use physiologic sensor system effective for clinical and research applications.

### SUMMARY OF THE INVENTION

Some of the above objects are achieved with a portable modular kiosk based physiologic sensor system for clinical and research applications configured to simultaneously utilize multiple sensors with cross checking and cross calculation of physiologic parameters.

The term “cross calculating” within the meaning of this application references the calculation of a physiologic parameter in which the input from at least two sensors that are coupled to two distinct standard input ports on the PC are utilized in the calculation of the parameter.

The term “cross checking” within the meaning of this application references the calculation of a physiologic parameter in which the input from at least two sensors that are coupled to two distinct standard input ports on the PC are utilized independently in the separate calculation of the parameter and these two values are utilized to arrive at a given parameter value.

One advantage of the present invention is that one embodiment permits the continuous monitoring of blood oxygenation and respiratory parameters particularly in small mammals.

These and other advantages of the present invention will be clarified in the description of the preferred embodiments taken together with the attached drawings in which like reference numerals represent like elements throughout.

### BRIEF DESCRIPTION OF THE DRAWINGS

FIG. 1 is a schematic view of a respiratory system for outpatient surgery performed under sedation level anesthesia according to a prior art system as described in U.S. Published Patent Application 2006-0264762;

FIG. 2 is a schematic view of a respiratory system for outpatient surgery performed under sedation level anesthesia according to a prior art system as described in U.S. Published Patent Application 2006-0264762;

FIG. 3 is a schematic view of a portable modular kiosk based physiologic sensor system for clinical and research applications configured to simultaneously utilize multiple sensors with cross checking and cross calculation of physiologic parameters in accordance with the present invention;

FIG. 4 is a schematic section view of tablet computer sample display of the portable modular kiosk based physiologic sensor system according to one aspect of the present invention;

FIG. 5 is a schematic view of a portable modular kiosk based physiologic sensor system for clinical and research applications configured to simultaneously utilize multiple sensors with cross checking and cross calculation of physiologic parameters in accordance with the present invention;

FIG. 6 is a schematic view of another portable modular PC based physiologic sensor system for clinical and research applications configured to simultaneously utilize multiple sensors with cross checking and cross calculation of physiologic parameters in accordance with the present invention; and

FIG. 7 is a schematic view of a portable modular PC based physiologic sensor system for clinical and research applications configured to simultaneously utilize permits the continuous monitoring of blood oxygenation and respiratory parameters particularly in small mammals in accordance with the present invention.

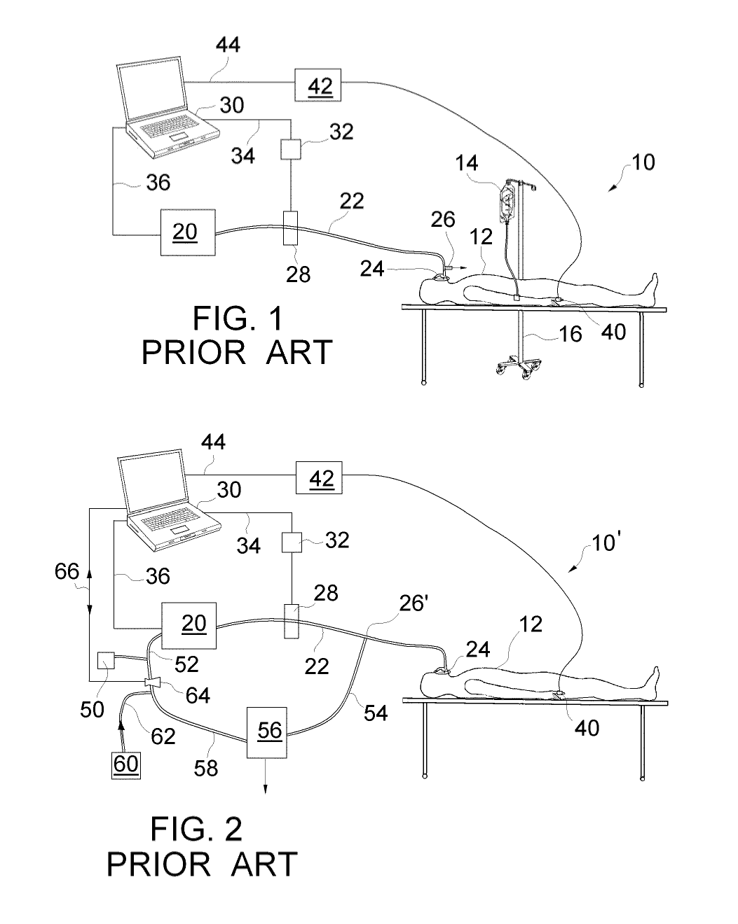

### DESCRIPTION OF THE PREFERRED EMBODIMENTS

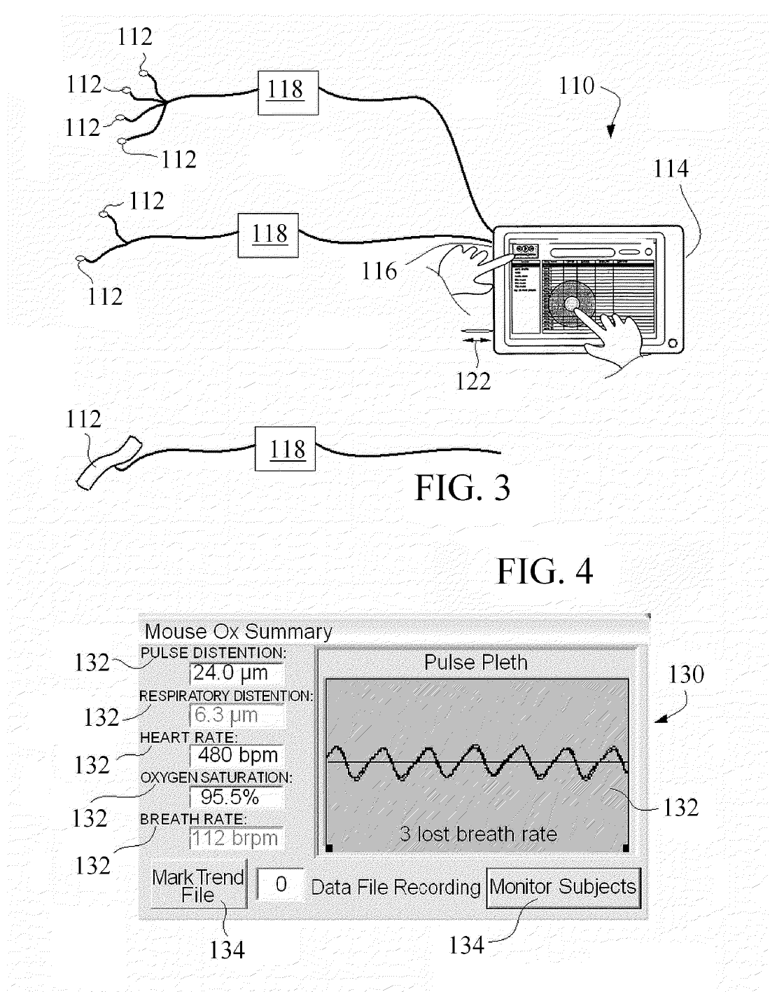

FIG. 3 is a schematic view of a portable modular kiosk based physiologic sensor system 110 for clinical and research applications configured to simultaneously utilize multiple sensors 112 with cross checking and cross calculation of physiologic parameters in accordance with the present invention.

One key component of the present invention is a PC computer 114 to which the multiple sensors 112 can be attached through conventional PC input ports 116. As noted above the input/output ports 116 of a personal computer 114 refer to the communications links through which the personal computers 114 send and receive information, which generally include serial ports, parallel ports, wireless links or connectors (such as WI-FL and Bluetooth), and universal serial bus (USB) ports 116. Where a physical connection is used (i.e. non-wireless), the USB port 116 will be the preferred connection for the present invention as several such input ports 116 are commonly provided on commercially available PC Computers 114.

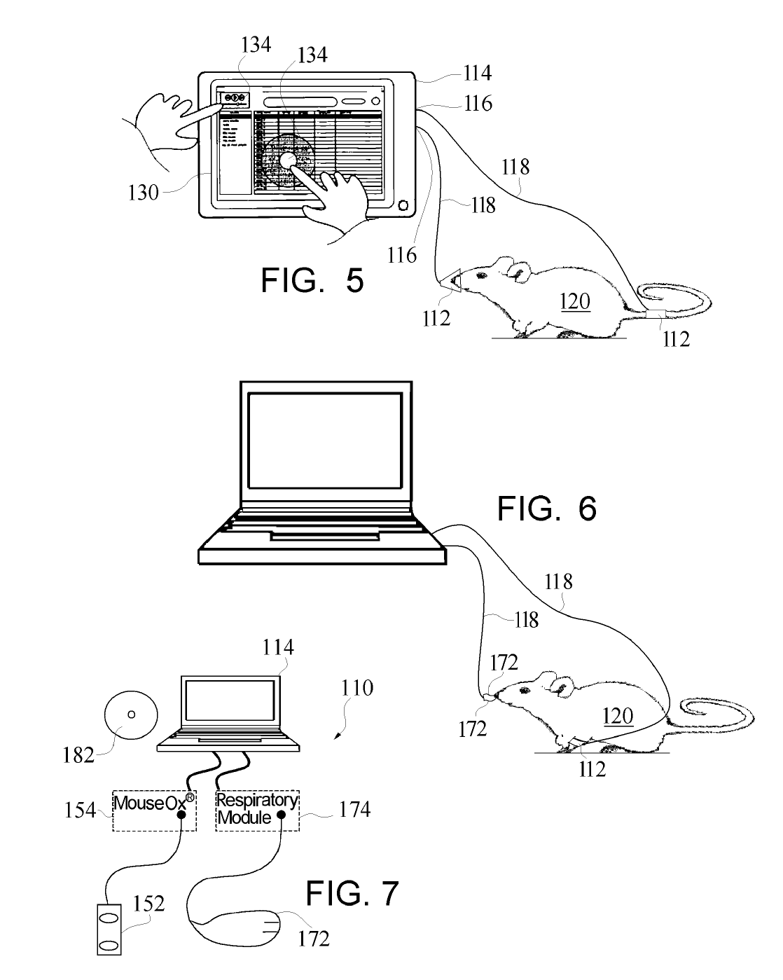

The PC computer 114 of the present invention is preferably a notebook computer 114 such as a tablet PC 114 as shown in FIGS. 3 and 5. As noted above a notebook computer 114 is an extremely lightweight PC that typically weighs less than six (6) pounds and is small enough to fit easily in a briefcase. Laptop computers and tablet PCs 114 are two types of notebook computers 114 for use in the present invention, wherein all of the interface hardware needed to operate the notebook computer 114, such as parallel and serial ports, graphics card, sound channel, etc., are built in to a single unit. The notebook computers 114 for implementing the present invention contain batteries to facilitate operation without a readily available electrical outlet.

In one embodiment of the present invention the system 110 according to the present invention is designed primarily for clinical applications. In the clinical application of the present invention the PC 114 is in the form of a Tablet PC 114, also called a panel computer, that incorporates a touch-screen 130 or “graphics tablet/screen hybrid technology” technology that allows the user to operate the computer with a stylus or digital pen 122, or a fingertip, instead of a keyboard or mouse. A joystick may be incorporated into the tablet 14 as a separate user input device.

In the clinical application of the present invention the Tablet PC 114 is operated in Kiosk mode. The Tablet PC 114 is particularly well suited to operate in Kiosk mode in light of the built in user interface provided with the tablet PC 114. One simple method of operating in Kiosk mode with the Tablet PC 114 is for the operating software to be launched at system start up. In a Microsoft Window® environment this is easily accomplished by dragging the operating software into the startup menu whereby it will be launched automatically at system startup. In this manner the system 110 is operable with simply a push of the on-off button.

One critical component of the present invention is a series of physiologic sensors 112 that are configured to be coupled to an input port 116 of the PC 14 such as through a USB port or through a wireless connection 118. Each sensor 112 is configured to measure a parameter related to a physical characteristic of a living subject, such as a human or animal, in particular a small mammal such as a mouse.

The types of physiologic sensors 112 include, for example, blood pressure sensors 112, blood flow sensors 112, blood glucose sensors 112, blood cholesterol sensors 112, heart sound sensors 112, EMG sensors 112, EEG sensors 112, EKG sensors 112, EOG sensors 112, pulse sensors 112, oxygenation sensors 112, pulse-oximetry sensors 112, blood perfusion sensors 112, respiration sensors (both pressure, flow and rate) 112, temperature sensors 112, additional blood gas sensors (such as nitrogen partial pressure, carbon dioxide partial pressure, carbon monoxide partial pressure, oxygen partial pressure, and pH level) 112, motion sensors 112, strain gauges 112, body position sensors 112, limb motion sensors 112 and the like.

For example, the MouseOx® sensor sold by the assignee of the present invention is one representative example of a sensor 112 for use in the system according to the present invention.

The sensors 112 will typically include an analog to digital converter (shown in coupling 118) between the sensor 112 elements that are coupled to the subject 120 and the PC coupling or port 116. Additionally the sensors 112 will occasionally need power. The sensors 112 may be plugged into a power supply through a separate power cord. However, in one aspect of the present invention separate power supply is incorporated into the sensor 112 to allow for elimination of the external power coupling and the need for a “close by” source of power, allowing the system to be particularly well suited for “field” operation. A separate method of eliminating the external power supply in the sensors 112 is to design the sensors 112 to draw power from the PC 114 through the coupling 118 to the PC 114. The elimination of the external power supply on the sensors 112 will assist in the portability of the system 110.

The modular aspect of the system 110 in accordance with the present invention is that a plurality of sensors 112 is provided for use in the system 110. It is anticipated that the user (clinician or researcher) will utilize a plurality of sensors 112 at the same time for any given subject 120 as shown in FIGS. 3 and 5. It is expected that these sensors 112 can be easily plugged into and removed from the PC 114. The PC 114 of the system 110 will recognize which sensors 112 are coupled to the input ports 116 and will configure the display 130 to display all of the relevant parameters at a designated display 132. The particular designated parameters display 132 that are calculated by the collection of sensors 112 (whether the parameters are calculated by individual sensors 112, cross checked with multiple sensors 112 or cross calculated from the output of two or more sensors 112). The display 130 can also include touch screen “buttons” or controls 134 for operation of the system 110.

Another aspect of the system 110 of the present invention is the cross checking of physiologic parameters determined by the system 110. In other words, for cross checking of a parameter, the calculation of the physiologic parameter uses the input from at least two sensors 112 that are coupled to two distinct standard input ports 116 on the PC 114 wherein these inputs are utilized independently in the separate calculation of the parameter and these two values are utilized to arrive at a given parameter value.

The concept of cross checking may be explained better by way of example. Breath rate of a subject 120 can be determined by both a ventilation based pressure sensor 112 and from a pulse oximeter 112. In this example the ventilation based sensor 112 is a respiratory sensor and the pulse oximeter is a cardiac sensor 112. Regardless the breath rate can be calculated by both sensors 112 independently and the Cross checking will use these two values in some fashion.

The use rules for cross checking may be that the dominant measurement rules. For example, the ventilation based sensor 112 can be considered a more accurate and robust measurement for this particular parameter, whereby the cross checking operates by having this be the dominant value. In other words if the ventilation based sensor 112 calculates the respiratory rate with a high degree of confidence then this is the value that is reported for this parameter and the pulse oximeter 112 reading or breath rate calculation is used if the respiratory sensor is not a confident reading. Further, the dominant reading can be compared to the less dominant reading and if there is a difference then the system can record that their may be an error in the non-dominant sensor. This can be important information since if the pulse oximeter is way off in the calculation of the breath rate then other readings may also be suspect. This cross checking system can thus be used as a sensor checking feature.

The use rules for cross checking may be an averaging of two calculated values to obtain a final parameter value, together with a checking of how far apart the two independent values are. The greater the variance in the independently obtained values the less confidence the system 110 should have in the accuracy of the final value. A threshold for the amount of difference that is acceptable can be utilized for each particular cross checked parameter.

The use rules for cross checking may include the use of threshold values and a discarding of those independently calculated parameter values that are outside the threshold. If both calculated parameters are outside the threshold then the system 110 can indicate that no value was calculated, or the system 110 may use the last known calculated parameter to display. If both calculated parameters are outside the threshold then the system 110 can further use one of the systems or methodologies described above such as averaging or dominant sensor rules.

The cross checking may be associated with more than two sensors 112, but the concept is not significantly different with three or more sensors 112 used to form a cross checked parameter.

Another aspect of the system of the present invention is the cross calculating of physiologic parameters determined by the system 110 wherein this defines the calculation of a physiologic parameter in which the input from at least two sensors 112 that are coupled to two distinct standard input ports 116 on the PC 114 are utilized in the calculation of the parameter. Cross calculation differs from cross checking in that the particular physiologic parameter cannot be calculated without the input from the two associated sensors.

An example of a cross calculated parameter is blood oxygenation/tidal volume. This parameter requires input from a pulse oximeter, for example, and a respiratory sensor. The present system contemplates one or more cross calculated parameters being made available to the user when two or more sensors are coupled to the PC. Pulse distention/tidal volume and breath distention/tidal volume would also represent cross calculated parameters available with the present invention that may be useful to researchers.

The cross calculating may be associated with more than two sensors 112, but the concept is not significantly different with three or more sensors 112 used to form a cross calculated parameter. Further the cross calculated parameters need not be simple ratios as presented in the example, but any combination of other parameters is contemplated.

FIGS. 6 and 7 illustrate one particular implementation of the present invention wherein the system 110 of FIGS. 6 and 7 permits the continuous monitoring of blood oxygenation and respiratory parameters in subjects, particularly small mammals such as mice and rats. These schematics illustrate the basic architecture of the system. For blood parameters, the system uses the MouseOx™ pulse oximeter 152 (a type of sensor 112) to measure arterial oxygen saturation, heart rate, pulse distention (which is a quantification of local blood flow) and breath distention (which is a quantification blood flow caused by breathing effort). All measurements are made from this simple foot wrap sensor 152.

Respiratory measurements are made using a simple dual-lumen cannula 172 in which one leg of the cannula provides supplemental oxygen to the patient or subject 120, while the other is used to assess a number of different respiratory parameters, including breath rate, gross tidal volume, gross minute ventilation, Tidal Volume/Breath Distention (in conjunction with the oximetry) and a number of breath timing parameters.

Both the oximetry 154 and respiratory 174 modules of the sensors 112 are controlled by software 182 loaded onto the PC 114, which is preferably a standard Windows®-based PC. Both modules 154 and 174 will connect to the PC 114 via USB ports. All parametric data can be saved to files, and select raw parameters can be accessed directly in analog form and fed into a recording device of the user's choosing.

The representative example of FIGS. 6 and 7 has been built and has the following characteristics. The subject or patient interface uses and foot wrap oximeter sensor 152 and associated module 154 and utilizes a Salter Labs dual-lumen nasal cannula with one lumen delivers supplemental oxygen and the other lumen is used by the system to measure airflow.

In the sample built the computer requirements include a PC with Pentium®-class processor (Pentium 1 GHz or higher recommended). The PC included a CD-ROM drive with a VGA or higher resolution monitor (Super VGA recommended). The operating system may be Windows® 2000, XP or Vista. The proposed memory was 512 MB RAM with 10 MB Hard-Drive Space for program (which does not include data files). The preferred Minimum Screen Resolution is 1024 by 768 pixels. The Communications for the PC included at least two USB ports (the oximeter module 154 and Respiratory Module 174 each require one 2.0 USB port). The designated Operating Wall Voltage is 100-240 VAC@ 50-60 Hz, with the Device Operating Voltage at 12 VAC (wall transformer) and the Max Operating Current being about 500 mA for the pulse oximer 154 and about 200 mA for the Respiratory Module 174.

The Analog Output Capabilities include Signals Available for Analog Output in the form of oximeter Infrared Pulse Waveform and Respiratory Module Airflow Waveform. The Signal Synchrony/Timing is Real Time and the Output Connector is a BNC male. The Signal Range/Characteristic for the waveforms is −5 to +5 volts.

As described above the oximeter 154 and Respiratory 174 Module to operate simultaneously under a single program, wherein the madules could have separate display windows, however it is anticipated that both windows can be displayed separately or together per user choice. The Display Window Update Rate is about 720 msec.

It is expected that the display includes a Continuous Waveforms for the Pulse Photoplethysmograph. The display further includes Parameter Strip Charts including: Pulse Rate (bpm), Pulse Distention (μm); Oxygen Saturation (%); Breath Distention (μm). The Respiratory Module Screen Display includes a Continuous Waveforms for Airflow. This display portion includes Parameter Strip Charts including: Breath Rate (brpm), Gross Tidal Volume [integral of pressure waveform], Gross Minute Ventilation [gross tidal volume*breath rate], Gross Tidal Volume/Breath Distention.

The system includes Data Storage/File Saving wherein the Data Files Formats are User-Selectable. Preferably it is a Single file for both Oximeter and Respiratory Module parameters. The system will provide a Default file name and location, but this is changeable with Windows file saving utility box. The system provides for Data Synchrony, wherein the Airflow Waveform, Infrared Pulse Waveform and all parameters will be stored to a file such that they are all synchronous with one another within +/−1 sample. The data storage rate is 300 hz.

The system stored parameters include the oximeter Parameters noted above, the Infrared Pulse Waveform, the Pulse Rate (bpm), Pulse Distention (μm), Oxygen Saturation (%), Breath Distention (μm), Error Codes for improper data and file Marker (0 to max of 50). The Respiratory Parameters include Airflow Waveform, Breath Rate (brpm), Inspiration Time (sec), Exhalation Time (sec), Breath Period (sec), Inspiration Time/Breath Period [Ti/Ttot], Gross Tidal Volume [integral of pressure waveform], Gross Minute Ventilation [gross tidal volume*breath rate], Gross Tidal Volume/Breath Distention.

The system can include an Apnea Alarm Indicator (0 or 1) and selected Parameter Alarms. The Alarm Types include Low and high threshold, wherein Low threshold activates when parameter crosses BELOW user-set threshold, and High threshold activates when parameter crosses ABOVE user-set threshold. In the event of an Alarm Signal the Pertinent strip chart color change and there is an Audible alarm with user-selectable silence.

The Pulse oximeter alarm parameters include: Pulse Rate (bpm), Pulse Distention (μm), Oxygen Saturation (%), Breath Distention (μm). The Airflow Module Alarm Parameters include Breath Rate (brpm), Peak to peak amplitude of Airflow,

The system can include an Apnea Alarm which can be a Two-state threshold alarm wherein the Alarm activates when apnea count exceeds user-set threshold and the Alarm can activate on either # of missed breaths OR apnea duration. The Alarm Signal may be that the Pertinent strip chart color change and/or an Audible alarm with user-selectable silence. The Alarm Limit Options would include the # of Missed Breaths to trigger the alarm, such as 2, 3, 4 or 5. Additionally the Apneic Duration can be set for 5, 10 and 15 sec.

The system shown in FIGS. 6 and 7 described above provide for the direct measure of animal respiration through the nasal canella 172. Further the integration of the pulse oximeter with the respiration allows the researcher or user to obtain the work of breathing which can be viewed as the pulse distension/tidal volume. The system provides for a duel or cross checking of the breath rate from the two sensors.

There are various modifications within the spirit of the present invention. For example for small mammels such as mice the nose cone of FIG. 5 can be replaced with a finger cot with a slit for a breathing hole to capture all of the gas. Patient or subject flow rates can be estimated from leak calculations. The invention contemplates a small orifice plate in the patient isoflurane connector for measuring flow without needing an external leak. A further modification of the present invention is to integrate the oximetry sensors into the nose cone to form an integrated multi sensor component.

In short the present invention provides a tool for clinicians, researchers, caregivers, educators and manufacturers that can be used in a number of distinct applications and although the present invention has been described with particularity herein, the scope of the present invention is not limited to the specific embodiments disclosed.

It will be apparent to those of ordinary skill in the art that various modifications may be made to the present invention without departing from the spirit and scope thereof. The scope of the invention is not to be limited by the illustrative examples described above. The scope of the present invention is defined by the appended claims and equivalents thereto.

What is claimed is:

1. A portable modular PC based physiologic sensor system for clinical and research applications configured to simultaneously utilize multiple sensors configured for the continuous monitoring of blood oxygenation and respiratory parameters.

2. The PC based physiologic sensor system according to claim 1 wherein the system includes a notebook PC.

3. The PC based physiologic sensor system according to claim 2 wherein the notebook PC is a tablet PC.

4. The PC based physiologic sensor system according to claim 3 wherein the sensors include at least two sensors that are configured to be simultaneously coupled to the tablet PC through a USB port connection.

5. The PC based physiologic sensor system according to claim 4 wherein the system is configured for operation on small mammals.

6. The PC based physiologic sensor system according to claim 5 wherein the sensors include at least one photoplythosmographic pulse oximeter.

7. A portable modular physiologic sensor system comprising a PC which is configured to simultaneously utilize multiple sensors with cross checking of at least one respiratory physiologic parameter, wherein the cross checking includes the calculation of the respiratory physiologic parameter using the input from at least two sensors that are coupled to two distinct standard input ports on the PC, wherein these inputs are utilized independently in the separate calculation of the cross checked respiratory parameter and these two independently calculated values of the cross checked parameter are utilized to arrive at a given cross checked parameter value.

8. The modular physiologic sensor system according to claim 7 wherein the system includes a notebook PC.

9. The modular physiologic sensor system according to claim 8 wherein the notebook PC is a tablet PC.

10. The modular physiologic sensor system according to claim 9 wherein the sensors include at least two sensors that are configured to be simultaneously coupled to the tablet PC through a USB port connection.

11. The modular physiologic sensor system according to claim 10 wherein the system is configured for operation on small mammals.

12. The modular physiologic sensor system according to claim 11 wherein the sensors include at least one pulse oximeter.

13. A portable modular physiologic sensor system comprising a PC which is configured to simultaneously utilize multiple sensors with cross calculation of at least one physiologic parameter wherein the cross calculation of a physiologic parameter utilizes the input from at least two sensors that are coupled to two distinct standard input ports on the PC.

14. The modular physiologic sensor system according to claim 13 wherein the system includes a notebook PC.

15. The modular physiologic sensor system according to claim 14 wherein the notebook PC is a tablet PC.

16. The modular physiologic sensor system according to claim 15 wherein the sensors include at least two sensors that are configured to be simultaneously coupled to the tablet PC through a USB port connection.

17. The modular physiologic sensor system according to claim 16 wherein the system is configured for operation on small mammals.

18. The modular physiologic sensor system according to claim 17 wherein the sensors include at least one pulse oximeter.

19. The modular physiologic sensor system according to claim 13 wherein a PC which is configured to simultaneously utilize multiple sensors with cross checking of at least one physiologic parameter, wherein the cross checking includes the calculation of the physiologic parameter using the input from at least two sensors that are coupled to two distinct standard input ports on the PC, wherein these inputs are utilized independently in the separate calculation of the cross checked parameter and these two independently calculated values of the cross checked parameter are utilized to arrive at a given cross checked parameter value.

20. The modular physiologic sensor system according to claim 14 wherein the PC is a tablet PC.
